SEQUENCE LISTING (11 sequences)

SEQ ID NO: 1 - DNA, 25 bp, Artificial Sequence
  Description of Artificial Sequence Synthetic oligonucleotide
atcgagagatagtggtcaccatcag

SEQ ID NO: 2 - DNA, 19 bp, Artificial Sequence
  Description of Artificial Sequence Synthetic primer
ccagtgccacaccgttgaa

SEQ ID NO: 3 - DNA, 21 bp, Artificial Sequence
  Description of Artificial Sequence Synthetic primer
tccccagatgatgcctttgtt

SEQ ID NO: 4 - DNA, 20 bp, Artificial Sequence
  Description of Artificial Sequence Synthetic primer
agcggccatttccattgccc

SEQ ID NO: 5 - DNA, 20 bp, Artificial Sequence
  Description of Artificial Sequence Synthetic primer
ccatgcccagagggtggttg

SEQ ID NO: 6 - DNA, 21 bp, Artificial Sequence
  Description of Artificial Sequence Synthetic primer
acagtggagctagaattggac

SEQ ID NO: 7 - DNA, 21 bp, Artificial Sequence
  Description of Artificial Sequence Synthetic primer
acttcccgaccaaggactttg

SEQ ID NO: 8 - DNA, 21 bp, Artificial Sequence
  Description of Artificial Sequence Synthetic primer
tgtggaggtcttggacttgga

SEQ ID NO: 9 - DNA, 21 bp, Artificial Sequence
  Description of Artificial Sequence Synthetic primer
tcctcagtcattctccccaaa

SEQ ID NO: 10 - DNA, 20 bp, Artificial Sequence
  Description of Artificial Sequence Synthetic primer
gcacagtcaaggccgagaat

SEQ ID NO: 11 - DNA, 20 bp, Artificial Sequence
  Description of Artificial Sequence Synthetic primer
gccttctccatggtggtgaa

(19) United States
(12) Patent Application Publication
(10) Pub. No.: US 2020/0085853 A1
(43) Pub. Date: Mar. 19, 2020

(54) TARGETING NR3B1 FOR TREATMENT OF NASH

(71) Applicant: University of Southern California, Los Angeles, CA (US)

(72) Inventor: Bangyan Stiles, Los Angeles, CA (US)

(21) Appl. No.: 16/515,970

(22) Filed: Jul. 18, 2019

Related U.S. Application Data
(60) Provisional application No. 62/701,420, filed on Jul. 20, 2018.

(51) Int. Cl. A61K 31/7105; C07K 16/28; C08G 69/02; C12N 15/113

(52) U.S. Cl. CPC A61K 31/7105; C07K 16/28; C12N 2310/14; C12N 15/113; C08G 69/02

(57) ABSTRACT

Provided herein are methods for one or more of: inhibiting the development of non-alcoholic steatohepatitis (NASH), non-alcoholic liver steatosis (NAFLD), fatty liver disease, liver fibrosis, hepatocellular carcinoma; blocking the biosynthesis of fatty acids and triglycerides; inhibiting de novo lipogeneis. The method comprises administering an effective amount of an agent that interferes with the binding of the estrogen receptor related receptor (NR3B) to a NR3B target to a subject in need thereof.

### CROSS-REFERENCE TO RELATED PATENT APPLICATION

This application claims priority under 35 U.S.C. § 119(e) to U.S. Provisional Application No. 62/701,420, filed Jul. 20, 2018, which is incorporated herein by reference in its entirety.

### STATEMENT OF GOVERNMENT SUPPORT

This invention was made with government support under the Grant No. R01CA154986-01, awarded by National Institutes of Health (NIH). The government has certain rights in the invention.

### BACKGROUND

The prevalence of liver steatosis is about 25% in the general population and as high as 70% in the obese population and those with type 2 diabetes. Without effective interventions, non-alcoholic liver steatosis (NAFLD) is in line to become the leading cause of liver transplantation in the next 10 years.¹ While moderate steatosis is benign and reversible, when compounded with inflammation and hepatocellular damage in non-alcoholic steatohepatitis (NASH), the prognosis to develop fibrosis, cirrhosis and even hepatocellular carcinoma is significantly increased. There are no currently FDA approved therapy for fatty liver or NASH treatment, though improving overall insulin resistance have been shown to have an effect on liver steatosis. Novel targets and therapy are in urgent need for NAFLD and NASH treatment. This disclosure satisfies this need.

### SUMMARY OF THE DISCLOSURE

The prevalence of liver steatosis is about 25% in the general population and as high as 70% in the obese population and those with type 2 diabetes.^{2,3} This number is expected to increase with the coming years in line with the predicted increased of obesity prevalence. Without effective interventions, non-alcoholic liver steatosis (NAFLD) is in line to become the leading cause of liver transplantation in the next 10 years.¹ While moderate steatosis is benign and reversible, when compounded with inflammation and hepatocellular damage in non-alcoholic steatohepatitis (NASH), the prognosis to develop fibrosis, cirrhosis and even hepatocellular carcinoma is significantly increased. There are no currently FDA approved therapy for fatty liver or NASH treatment, though improving overall insulin resistance have been shown to have an effect on liver steatosis. A recent phase I study demonstrated that targeting the nuclear receptor farnesoid X nuclear receptor (FXR) with a synthetic variant of the natural bile acid can improve the histological features of NASH, however with increased low-density lipoprotein as an unwanted side effect.⁴ Novel targets and therapy are in urgent need for NAFLD and NASH treatment.

Lipids accumulate in hepatocytes through several routes.⁵ Beside dietary lipids, fatty acids released from adipose tissue also serves as a source of lipids for liver uptake, particularly with insulin resistance. De novo lipogenesis resulting from elevated insulin secretion also serves as a source of lipid for hepatocytes and is currently considered an important contributing factor for NAFLD.⁶ Disposal of hepatic lipids depends on fatty acid oxidation as well as triglyceride secretion as lipoprotein particles. Alterations in any of these processes may shift the balance of lipid homeostasis in hepatocytes, leading to steatosis, though no clear mechanism has been established for the pathogenesis of steatosis.⁵ Mitochondria, which participate in multiple metabolic processes is proposed to play a role in the pathogenesis of NAFLD.⁷ Production of reactive oxygen species (ROS) due to accelerated or dysfunctional oxygen consumption by the mitochondria can propagate NASH development.⁸ Mouse studies that manipulate mitochondrial proteins such as the mitochondrial trifunctional protein, the mitochondrial and nuclear forms of sirtuins support a role of mitochondrial in NAFLD and NASH pathogenesis.^{9,10} NR3B, also known as the estrogen receptor related receptor (ERR), is a nuclear transcriptional activator for genes involved in mitochondrial bioenergetics and a master regulator of mitochondrial function.¹² NR3B knockouts are resistant to high-fat diet induced obesity.¹³ In a NAFLD/NASH model where a negative regulator of insulin signal Pten (phosphatase and tensin homologue deleted on chromosome 10) is deleted specifically in the liver,¹⁴⁻¹⁷ Applicants found that NR3B is robustly induced. Applicants also determined that activation of insulin/PI3K signaling pathway induces NR3B to stimulate increased mitochondrial biogenesis and upregulate oxygen consumption.

Applicants further discovered that inhibiting NR3B blocks the accumulation of lipid in the NAFLD/NASH model where PTEN is lost. In addition, the NR3B inhibition was also found to block NAFLD development induced by feeding of high carbohydrate diet (HCD). This effect of NR3B is due to its ability to block the biosynthesis of triglyceride in addition to inhibiting de novo lipogenesis. The results of this example further indicated a potential role of RXR as either cofactor or downstream target for NR3B regulated gene transcription. This example further established NR3B as a potential target for NAFLD/NASH treatment and elucidated novel signals that are regulated by NR3B in this process. As such, the results reported herein have significantly impact in liver disease and the development of NAFLD/NASH therapy.

Thus, in one aspect provided herein is a method for one or more of: inhibiting the development of non-alcoholic steatohepatitis (NASH), non-alcoholic liver steatosis (NAFLD), fatty liver disease, liver fibrosis, hepatocellular carcinoma; blocking the biosynthesis of fatty acids and triglycerides; inhibiting de novo lipogeneis; the method comprising, or alternatively consisting essentially of, or yet further consisting of, the administering an effective amount of an agent that interferes with the binding of the estrogen receptor related receptor (NR3B) to a NR3B target to a subject in need thereof. A non-limiting example of the NR3B target is one or more promoters of a NR3B target gene. Non-limiting examples of such include the NR3B target genes cytochrome C and/or MCAD.

In another aspect, the agent is selected from the group of: a small molecule, an anti-NR3B antibody, an NR3B-polyamide (NR3B-PA), an anti-NR3B inhibitory RNA, or siNR3B. An example of a small molecule includes XCT 790 (Sigma) or a prodrug, salt or solvate thereof.

In one aspect, the agent is NR3B-PA or an equivalent thereof or siNR3B or an equivalent thereof.

The agents can be administered in a composition comprising the agent and a carrier such as a pharmaceutically acceptable carrier. Administration can be in one or more doses and can be local in the region of the site of injury (e.g., the liver) or systemic. In a further aspect, the method further comprises administering another agent that treats the relative one or more diseases or conditions.

The methods are useful to treat any subject that expresses NR3B, e.g., a mammal such as a human patient.

### BRIEF DESCRIPTION OF THE DRAWINGS

FIGS. 1A-1B: FIG. 1A shows an imidazole and pyrrole containing polyamide with the same DNA binding sequence occupancy as NR3B. FIG. 1B shows a gel mobility shift assay with biotin labeled DNA probe containing an estrogen-related response element (ERRE).

FIGS. 2A-2B: FIG. 2A shows that at the minimum effective dose for DNA gel mobility shift assay (0.1 μM), NR3B-PA reduced both basal and maximal oxygen consumption rate (OCR) in a Seahorse assay whereas the control PA not matched to the sequence (MM-PA) did not. This effect is similar to what is observed with XCT 790 (also shown), a commercially available NR3B inverse agonist. FIG. 2B shows that increasing the dose of PA led to dose dependent reduction in OCR, similar to what is observed with knockdown of NR3B with siRNA.

FIGS. 3A-3C: FIG. 3A shows results of using luciferase reporter containing the DNA binding element for NR3B. The results show that NR3B-PA treatment inhibited the luciferase activity of the reporter whereas MM-PA did not at the same dose. FIG. 3B shows in both mouse and human hepatocytes, NR3B-PA treatment downregulated the expression of cytochrome C, a target gene of NR3B. FIG. 3C shows RNA seq analysis displays that more than 90% of the genes downregulated by NR3B-PA treatment are also regulated by siNR3B, confirming specificity of NR3B-PA for NR3B.

FIGS. 4A-4D: FIG. 4A shows the effect of polyamide treatment on liver steatosis in Pten-null mice. Using this NR3B-PA to block NR3B transcriptional activity, 1.5-month old Pten-null mice were treated with 25 nmole polyamide every four days for 1 month and results showed that inhibiting NR3B with polyamide blocked liver steatosis. FIG. 4B shows body weight and triglyceride quantification results of NR3B-PA-treated Pten null mice vs. vehicle and wildtype. FIG. 4C shows immunoblotting analysis using liver extracts from control, vehicle treated and NR3B-PA treated Pten null mice. FIG. 4D shows expression of various mRNAs in wt, pten-null (Pm) and pten-null polyamide treated (Pm+PA) liver tissue.

FIGS. 5A-5C: FIG. 5A is a heat map that shows lower (blue vs. red) expression of genes involved in triglyceride synthesis in siNR3B treated cells vs. controls; a scheme for the biosynthesis of triglycerides indicating the role of these genes in triglyceride synthesis is present on the right. FIG. 5B shows results of a qPCR analysis that confirm downregulation of DGAT2 and AGAPT3 upon siNR3B treated samples vs control. FIG. 5C shows NR3B-PA treatment also induced similar downregulation of the same genes in hepatocytes.

FIGS. 6A-6B: FIG. 6A shows that treatment with Bexrotene, an agonist for RXR leads to induction of Fasn, DGAT2 and AGPAT3. FIG. 6B shows the effect of bexarotene treatment on the effects of MR3B-PA treatment on OCR production.

FIGS. 7A-7B: FIG. 7A shows the triglyceride levels in the liver of HCD fed mice, in control, mismatched-PA (MM-PA), NR3B-PA (PA), NC (normal chow diet), and NC+PA treated mice. Scr-PA is the MM-PA, and it stands for scrambled.

FIG. 7B shows the effects of NR3B inhibition on reversing the phenotype of fatty liver, particularly at the NASH stage using the 7.5 months old Pten null mice. NR3B-PA treatment reversed the severe NASH phenotype that is observed with PTEN loss at this age.

FIG. 8 shows the negative correlation of PTEN expression vs. NASH.

### DETAILED DESCRIPTION OF THE DISCLOSURE

Throughout this disclosure, various publications, patents and published patent specifications are referenced by an identifying citation. The disclosures of these publications, patents and published patent specifications are hereby incorporated by reference into the present disclosure in their entirety to more fully describe the state of the art to which this invention pertains.

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2^{nd} edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R. I. Freshney, ed. (1987)).

As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compounds, compositions and methods include the recited elements, but not exclude others. “Consisting essentially of” when used to define compounds, compositions and methods, shall mean excluding other elements of any essential significance to the combination. Thus, a composition consisting essentially of the elements as defined herein would not exclude trace contaminants, e.g., from the isolation and purification method and pharmaceutically acceptable carriers, preservatives, and the like. “Consisting of” shall mean excluding more than trace elements of other ingredients. Embodiments defined by each of these transition terms are within the scope of this technology.

All numerical designations, e.g., pH, temperature, time, concentration, and molecular weight, including ranges, are approximations which are varied (+) or (−) by increments of 1, 5, or 10%. It is to be understood, although not always explicitly stated that all numerical designations are preceded by the term “about.” It also is to be understood, although not always explicitly stated, that the reagents described herein are merely exemplary and that equivalents of such are known in the art.

As used herein, the term “reporter” means an element on or within an isolated cell having a characteristic (e.g., activity, expression, localization, interaction, modification, etc.) which is one or more of: dependent upon, correlates with, or activated by physiological changes or conditions of the cell.

“NASH” intends non-alcoholic steatohepatitis which is a type of NAFLD.

“NAFLD” intends nonalcoholic fatty liver disease, a condition in which fat builds up in the liver. NASH and NAFLD are associated with inflammation and liver cell damage, along with fat in the liver.

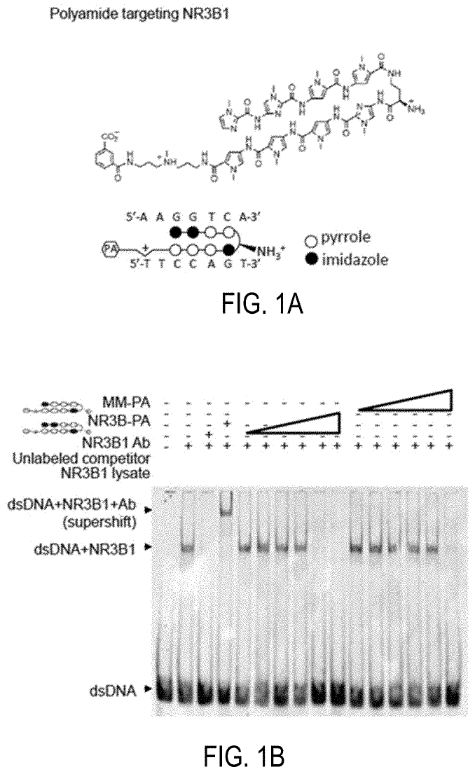

NR3B-PA is a polyamide that targets NR3B. An example of such is shown in FIG. 1A.

“Analogue” may refer to a structural analogue or functional analogue of a chemical compound. Structural analogues share similarity in chemical structure. Functional analogues share similarity in their physical, chemical, biochemical or pharmacological properties.

An “agent” intends a small molecule or molecules, or large molecule or molecules or biologic or biologics or any combination thereof, e.g., inhibitory RNA, a small molecule, or antibody, antibody fragment or modification thereof.

“Candidate agent” refers to a compound for the treatment of one or more of: NAFLD, NASH, fatty liver disease, liver fibrosis, cirrhosis, hepatocellular carcinoma, in a subject in need thereof.

“Pharmaceutically acceptable salt” refers to salts of a compound, which salts are suitable for pharmaceutical use and are derived from a variety of organic and inorganic counter ions well known in the art and include, when the compound contains an acidic functionality, by way of example only, sodium, potassium, calcium, magnesium, ammonium, and tetraalkylammonium; and when the molecule contains a basic functionality, salts of organic or inorganic acids, such as hydrochloride, hydrobromide, tartrate, mesylate, acetate, maleate, and oxalate (see Stahl and Wermuth, eds., “Handbook of Pharmaceutically Acceptable Salts,” (2002), Verlag Helvetica Chimica Acta, Zürich, Switzerland), for a discussion of pharmaceutical salts, their selection, preparation, and use.

Generally, pharmaceutically acceptable salts are those salts that retain substantially one or more of the desired pharmacological activities of the parent compound and which are suitable for in vivo administration. Pharmaceutically acceptable salts include acid addition salts formed with inorganic acids or organic acids. Inorganic acids suitable for forming pharmaceutically acceptable acid addition salts include, by way of example and not limitation, hydrohalide acids (e.g., hydrochloric acid, hydrobromic acid, hydroiodic acid, etc.), sulfuric acid, nitric acid, phosphoric acid, and the like.

Organic acids suitable for forming pharmaceutically acceptable acid addition salts include, by way of example and not limitation, acetic acid, trifluoroacetic acid, propionic acid, hexanoic acid, cyclopentanepropionic acid, glycolic acid, oxalic acid, pyruvic acid, lactic acid, malonic acid, succinic acid, malic acid, maleic acid, fumaric acid, tartaric acid, citric acid, palmitic acid, benzoic acid, 3-(4-hydroxybenzoyl) benzoic acid, cinnamic acid, mandelic acid, alkylsulfonic acids (e.g., methanesulfonic acid, ethanesulfonic acid, 1,2-ethane-disulfonic acid, 2-hydroxyethanesulfonic acid, etc.), arylsulfonic acids (e.g., benzenesulfonic acid, 4-chlorobenzenesulfonic acid, 2-naphthalenesulfonic acid, 4-toluenesulfonic acid, camphorsulfonic acid, etc.), glutamic acid, hydroxynaphthoic acid, salicylic acid, stearic acid, muconic acid, and the like.

Pharmaceutically acceptable salts also include salts formed when an acidic proton present in the parent compound is either replaced by a metal ion (e.g., an alkali metal ion, an alkaline earth metal ion, or an aluminum ion) or by an ammonium ion (e.g., an ammonium ion derived from an organic base, such as, ethanolamine, diethanolamine, triethanolamine, morpholine, piperidine, dimethylamine, diethylamine, triethylamine, and ammonia).

A solvate of a compound is a solid-form of a compound that crystallizes with less than one, one or more than one molecules of a solvent inside in the crystal lattice. A few examples of solvents that can be used to create solvates, such as pharmaceutically acceptable solvates, include, but are not limited to, water, C₁-C₆ alcohols (such as methanol, ethanol, isopropanol, butanol, and can be optionally substituted) in general, tetrahydrofuran, acetone, ethylene glycol, propylene glycol, acetic acid, formic acid, and solvent mixtures thereof. Other such biocompatible solvents which may aid in making a pharmaceutically acceptable solvate are well known in the art. Additionally, various organic and inorganic acids and bases can be added to create a desired solvate. Such acids and bases are known in the art. When the solvent is water, the solvate can be referred to as a hydrate. In some embodiments, one molecule of a compound can form a solvate with from 0.1 to 5 molecules of a solvent, such as 0.5 molecules of a solvent (hemisolvate, such as hemihydrate), one molecule of a solvent (monosolvate, such as monohydrate) and 2 molecules of a solvent (disolvate, such as dihydrate).

An animal, subject or patient for diagnosis or treatment refers to an animal such as a mammal, or a human, ovine, bovine, feline, canine, equine, simian, etc. Non-human animals subject to diagnosis or treatment include, for example, simians, murine, such as, rat, mice, canine, leporid, livestock, sport animals, and pets. In one aspect, the subject is a human. It is to be understood that the terms “subject” and “patient” are interchangeable.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. Unless specifically noted otherwise, the term “antibody” includes intact immunoglobulins and “antibody fragments” or “antigen binding fragments” that specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴M⁻¹ greater or at least 10⁵ M⁻¹ greater than a binding constant for other molecules in a biological sample). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

In terms of antibody structure, an immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopts a β-sheet conformation and the CDRs form loops which connect, and in some cases form part of, the β-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. An antibody that binds LHR will have a specific V_{H} region and the V_{L} region sequence, and thus specific CDR sequences. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, the term “vector” refers to a nucleic acid construct deigned for transfer between different hosts, including but not limited to a plasmid, a virus, a cosmid, a phage, a BAC, a YAC, etc. In some embodiments, plasmid vectors may be prepared from commercially available vectors. In other embodiments, viral vectors may be produced from baculoviruses, retroviruses, adenoviruses, AAVs, etc. according to techniques known in the art. In one embodiment, the viral vector is a lentiviral vector. It is to be understood that the vectors contain the necessary regulatory elements for replication or expression of the inserted polynucleotide, including for example promoters or enhancer elements.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors.

As used herein, the term “label” intends a directly or indirectly detectable compound or composition that is conjugated directly or indirectly to the composition to be detected, e.g., N-terminal histidine tags (N-His), magnetically active isotopes, e.g., ¹¹⁵-sn, ¹¹⁷Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g., radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component. Examples of luminescent labels that produce signals include, but are not limited to bioluminescence and chemiluminescence. Detectable luminescence response generally comprises a change in, or an occurrence of a luminescence signal. Suitable methods and luminophores for luminescently labeling assay components are known in the art and described for example in Haugland, Richard P. (1996) Handbook of Fluorescent Probes and Research Chemicals (6^{th} ed). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6^{th} ed.).

In another aspect, the fluorescent label is functionalized to facilitate covalent attachment to a cellular component present in or on the surface of the cell or tissue such as a cell surface marker. Suitable functional groups, include, but are not limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

A host cell can be a eukaryotic or a prokaryotic cell. “Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human.

“Prokaryotic cells” that usually lack a nucleus or any other membrane-bound organelles and are divided into two domains, bacteria and archaea. In addition to chromosomal DNA, these cells can also contain genetic information in a circular loop called on episome. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

As used herein, the term “detectable marker” refers to at least one marker capable of directly or indirectly, producing a detectable signal. A non-exhaustive list of this marker includes enzymes which produce a detectable signal, for example by colorimetry, fluorescence, luminescence, such as horseradish peroxidase, alkaline phosphatase, β-galactosidase, glucose-6-phosphate dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties, such detection may be accomplished by optical methods such as diffraction, surface plasmon resonance, surface variation , the contact angle change or physical methods such as atomic force spectroscopy, tunnel effect, or radioactive molecules such as ³² P, ³⁵S or ¹²⁵I.

As used herein, the term “purification label” refers to at least one marker useful for purification or identification. A non-exhaustive list of this marker includes His, lacZ, GST, maltose-binding protein, NusA, BCCP, c-myc, CaM, FLAG, GFP, YFP, cherry, thioredoxin, poly(NANP), V5, Snap, HA, chitin-binding protein, Softag 1, Softag 3, Strep, or S-protein. Suitable direct or indirect fluorescence marker comprise FLAG, GFP, YFP, RFP, dTomato, cherry, Cy3, Cy 5, Cy 5.5, Cy 7, DNP, AMCA, Biotin, Digoxigenin, Tamra, Texas Red, rhodamine, Alexa fluors, FITC, TRITC or any other fluorescent dye or hapten.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample. In one aspect, the expression level of a gene from one sample may be directly compared to the expression level of that gene from a control or reference sample. In another aspect, the expression level of a gene from one sample may be directly compared to the expression level of that gene from the same sample following administration of a compound.

As used herein, “homology” or “identical”, percent “identity” or “similarity”, when used in the context of two or more nucleic acids or polypeptide sequences, refers to two or more sequences or subsequences that are the same or have a specified percentage of nucleotides or amino acid residues that are the same, e.g., at least 60% identity, preferably at least 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein). Homology can be determined by comparing a position in each sequence that may be aligned for purposes of comparison. When a position in the compared sequence is occupied by the same base or amino acid, then the molecules are homologous at that position. A degree of homology between sequences is a function of the number of matching or homologous positions shared by the sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. The terms “homology” or “identical”, percent “identity” or “similarity” also refer to, or can be applied to, the complement of a test sequence. The terms also include sequences that have deletions and/or additions, as well as those that have substitutions. As described herein, the preferred algorithms can account for gaps and the like. Preferably, identity exists over a region that is at least about 25 amino acids or nucleotides in length, or more preferably over a region that is at least 50-100 amino acids or nucleotides in length. An “unrelated” or “non-homologous” sequence shares less than 40% identity, or alternatively less than 25% identity, with one of the sequences disclosed herein.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at www.cancer.gov, last visited on May 1, 2008. Typically, a patient is given a subsequent chemotherapy regimen because the patient did not show a positive clinical or sub-clinical response to the first line therapy or the first line therapy has stopped.

In one aspect, the term “equivalent” or “biological equivalent” of an antibody means the ability of the antibody to selectively bind its epitope protein or fragment thereof as measured by ELISA or other suitable methods. Biologically equivalent antibodies include, but are not limited to, those antibodies, peptides, antibody fragments, antibody variant, antibody derivative and antibody mimetics that bind to the same epitope as the reference antibody.

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.govicgi-bin/BLAST.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

A “composition” as used herein, refers to an active agent, such as an agent as disclosed herein and a carrier, inert or active. The carrier can be, without limitation, solid such as a bead or resin, or liquid, such as phosphate buffered saline.

“Administration,” “administering” and the like intends by any appropriate means, e.g., intravenously, orally, by suppository, inhalation, or other, an agent, composition or combination as described herein.

Administration or treatment in “combination” refers to administering two agents such that their pharmacological effects are manifest at the same time. Combination does not require administration at the same time or substantially the same time, although combination can include such administrations.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents disclosed herein for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. In general, one will desire to administer an amount of the compound that is effective to achieve a serum level commensurate with the concentrations found to be effective in vivo. These considerations, as well as effective formulations and administration procedures are well known in the art and are described in standard textbooks. Consistent with this definition and as used herein, the term “therapeutically effective amount” is an amount sufficient to treat a specified disorder or disease or alternatively to obtain a pharmacological response such as treatment of NASH or fatty liver disease.

As used herein, “treating” or “treatment” of a disease in a patient refers to (1) preventing the symptoms or disease from occurring in an animal that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of this technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, the term treatment excludes prevention or prophylaxis.

### Therapeutic Methods

Provided herein are methods for the treatment of one or more of: inhibiting the development of non-alcoholic steatohepatitis (NASH), non-alcoholic liver steatosis (NAFLD), fatty liver disease, liver fibrosis, hepatocellular carcinoma; blocking the biosynthesis of fatty acids and triglycerides; or inhibiting de novo lipogeneis; the method comprising, or alternatively consisting essentially of, or yet further consisting of, administering an effective amount of an agent that interferes with the binding of the estrogen receptor related receptor (NR3B) to a NR3B target to a subject in need thereof. In one aspect, the agent is labeled.

As described by Tremblay and Giguere, (2007) Nucl. Recept. Signal 5:e0009 doi:10.1621/nrs.05009, the NR3B subgroup includes three nuclear receptors referred to as ERRα (NR3B1, ERR1, ESRRA), ERRβ (NR3B2, ERR2, ESRRB) and ERRy (NR3B3, ERR3, ESRRG), respectively. The NR3B subgroup belongs to the larger NR3 class of nuclear receptors that includes the classic steroid hormone receptors for estrogens, androgens, progesterone, aldosterone and cortisol.

In one aspect, NR3B targets are the promoters of a NR3B target gene. Non-limiting examples of such include cytochrome C or Medium Chain Acyl-CoA Dehydrogenase (MCAD).

Non-limiting examples of agents for use in the methods are selected from the group of: an anti-NR3B antibody, an NR3B-polyamide (NR3B-PA) or an equivalent thereof, an anti-NR3B inhibitory RNA, or siNR3B.

In some embodiment, the antibody is a polyclonal or a monoclonal antibody. In a related embodiment, the monoclonal antibody is humanized or specisized. In another aspect, an antigen binding fragment of the antibody is utilized. In one aspect, the antibodies or antigen binding fragments are labeled with a detectable or a purification label.

Also provided are polynucleotides encoding the antibodies and antigen binding fragments that are optionally labeled with a detectable or purification label, wherein in one aspect, the detectable label is not a continguous naturally occurring nucleic acid. Also provided are host cells containing these polynucleotides and/or polypeptides and use of the cells for recombinant production of these compositions. The cells can be prokaryotic or eukaryotic. Further provided are host vector systems comprising the polynucleotides, e.g., a replication or another insertion vector. These compositions can be combined with a carrier, such as a pharmaceutically acceptable carrier and/or an adjuvant. Further provided are methods for generating these antibodies and antigen binding fragments.

Non-limiting examples of agents also include the siNR3B sequence or an equivalent thereof, as well at the NR3B-PA, and an equivalent thereof.

The methods can be modified by administration of one or more additional agents that is known for treatment of the one or more diseases or conditions. For example, one could combine the disclosed therapy with a chemotherapeutic for the treatment of hepatocellular carcinoma.

The methods as disclosed herein are not limited by the mode of administration, and include without limitation, locally to the site of disease or injury or systemically, e.g., orally, intravenously or by suppository. A subject in need thereof includes mammals, such as pets and veterinary animals, and human patients. As used herein, an effective amount intends an amount to reduce or alleviate the symptoms of the disease or disorder, and vary with the subject being treated, the age, the disease or disorder, and the treating physician. The treating physician or her assistant can determine when the method has been successful by observing symptoms.

When treating cancer, the agents can be administered as first line, second line, third line, fourth line of fifth line therapy.

In some refinements, the effective amount is between about 0.05 mg/kg to about 20 mg/kg of body weight of the subject. In some refinements, the effective amount is between about 0.1 mg/kg to about 10 mg/kg. In some refinements, the effective amount is between about 0.2 mg/kg to about 8 mg/kg. In some refinements, the effective amount is between about 0.5 mg/kg to about 6 mg/kg. In some refinements, the effective amount is between about 1 mg/kg to about 5 mg/kg. In some refinements, the effective amount is between about 2 mg/kg to about 5 mg/kg. In some refinements, the effective amount is 2.5 mg/kg. In some refinements, the effective amount is 5 mg/kg.

In some refinements, the effective amount is administered once a day. In some refinements, the effective amount is administered twice a day. In some refinements, the effective amount is administered thrice a day. In some refinements, the effective amount is administered four times a day. In some refinements, the effective amount is administered continuously through infusion. In further embodiments, the effective amount is administered in the same or different dosage amount, and/or in the same or different routes of administration, and/or in the same or different formulation.

In some refinements, the effective amount is administered daily for about 2 days to about 7 days. In some refinements, the effective amount is administered daily for about 2 days to about 2 weeks. In some refinements, the effective amount is administered daily for about 1 week to about 1 month. In some refinements, the effective amount is administered daily for about 1 month to about 6 months. In some refinements, the effective amount is administered daily for about 6 months to about 1 year. In some refinements, the effective amount is administered daily for about 1 year to about 2 years. In some refinements, the effective amount is administered daily for about 2 years to about 5 years. In some refinements, the effective amount is administered daily for the lifetime of the subject. In further embodiments, the effective amount is administered in the same or different dosage amount, and/or in the same or different routes of administration, and/or in the same or different formulation.

### Methods of Selecting a Candidate Agent for Treatment

Also provided is a method for selecting a candidate agent for the treatment of one or more of inhibiting the development of non-alcoholic steatohepatitis (NASH), non-alcoholic liver steatosis (NAFLD), fatty liver disease, liver fibrosis, hepatocellular carcinoma; blocking the biosynthesis of fatty acids and triglycerides; or inhibiting de novo lipogeneis. One of skill in the art will test the ability of the candidate agent to interfere with the binding of NR3B to its target, in vitro or in vivo. If it inhibits binding, it is a candidate agent. One of skill in the art can compare the effectiveness against the agents identified herein for sole or combination use.

It is to be understood that “compound” as it recited in this method may refer to, but not be limited by, a small molecule, a macromolecule, or a biologic.

### Compositions

Compositions, including pharmaceutical compositions comprising the agents or compounds described herein can be manufactured by means of conventional mixing, dissolving, granulating, levigating, emulsifying, encapsulating, entrapping, or lyophilization processes. The compositions can be formulated in conventional manner using one or more physiologically acceptable carriers, diluents, excipients, or auxiliaries which facilitate processing of the compounds provided herein into preparations which can be used pharmaceutically.

The agents and compounds of the technology can be administered by parenteral (e.g., intramuscular, intraperitoneal, intravenous, ICV, intracisternal injection or infusion, subcutaneous injection, or implant), oral, by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration.

In one embodiment, this disclosure relates to a composition comprising a compound as described herein and a carrier.

In another embodiment, this disclosure relates to a pharmaceutical composition comprising a compound as described herein and a pharmaceutically acceptable carrier.

In another embodiment, this disclosure relates to a pharmaceutical composition comprising a therapeutically effective amount of a compound as described herein and a pharmaceutically acceptable carrier.

The pharmaceutical compositions for the administration of the compounds can be conveniently presented in dosage unit form and can be prepared by any of the methods well known in the art of pharmacy. The pharmaceutical compositions can be, for example, prepared by uniformly and intimately bringing the compounds provided herein into association with a liquid carrier, a finely divided solid carrier or both, and then, if necessary, shaping the product into the desired formulation. In the pharmaceutical composition the compound provided herein is included in an amount sufficient to produce the desired therapeutic effect. For example, pharmaceutical compositions of this disclosure may take a form suitable for virtually any mode of administration, including, for example, topical, ocular, oral, buccal, systemic, nasal, injection, infusion, transdermal, rectal, and vaginal, or a form suitable for administration by inhalation or insufflation.

For topical administration, the compounds can be formulated as solutions, gels, ointments, creams, suspensions, etc., as is well-known in the art.

Systemic formulations include those designed for administration by injection (e.g., subcutaneous, intravenous, infusion, intramuscular, intrathecal, or intraperitoneal injection) as well as those designed for transdermal, transmucosal, oral, or pulmonary administration.

Useful injectable preparations include sterile suspensions, solutions, or emulsions of the compounds provided herein in aqueous or oily vehicles. The compositions may also contain formulating agents, such as suspending, stabilizing, and/or dispersing agents. The formulations for injection can be presented in unit dosage form, e.g., in ampules or in multidose containers, and may contain added preservatives.

Alternatively, the injectable formulation can be provided in powder form for reconstitution with a suitable vehicle, including but not limited to sterile pyrogen free water, buffer, and dextrose solution, before use. To this end, the compounds provided herein can be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets, or capsules prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulfate). The tablets can be coated by methods well known in the art with, for example, sugars, films, or enteric coatings.

Compositions intended for oral use can be prepared according to any method known to the art for the manufacture of pharmaceutical compositions, and such compositions may contain one or more agents selected from the group consisting of sweetening agents, flavoring agents, coloring agents, and preserving agents in order to provide pharmaceutically elegant and palatable preparations. Tablets contain the compounds provided herein in admixture with non-toxic pharmaceutically acceptable excipients which are suitable for the manufacture of tablets. These excipients can be for example, inert diluents, such as calcium carbonate, sodium carbonate, lactose, calcium phosphate or sodium phosphate; granulating and disintegrating agents (e.g., corn starch or alginic acid); binding agents (e.g., starch, gelatin, or acacia); and lubricating agents (e.g., magnesium stearate, stearic acid, or talc). The tablets can be left uncoated or they can be coated by known techniques to delay disintegration and absorption in the gastrointestinal tract and thereby provide a sustained action over a longer period. For example, a time delay material such as glyceryl monostearate or glyceryl distearate can be employed. They may also be coated by the techniques well known to the skilled artisan. The pharmaceutical compositions of the technology may also be in the form of oil-in-water emulsions.

Liquid preparations for oral administration may take the form of, for example, elixirs, solutions, syrups, or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives, or hydrogenated edible fats); emulsifying agents (e.g., lecithin, or acacia); non-aqueous vehicles (e.g., almond oil, oily esters, ethyl alcohol, cremophore™, or fractionated vegetable oils); and preservatives (e.g., methyl or propyl-p-hydroxybenzoates or sorbic acid). The preparations may also contain buffer salts, preservatives, flavoring, coloring, and sweetening agents as appropriate.

### Use of Compounds for Preparing Medicaments

The agents or compounds and compositions of the present invention are also useful in the preparation of medicaments to treat a variety of pathologies as described herein. The methods and techniques for preparing medicaments of a composition are known in the art. For the purpose of illustration only, pharmaceutical formulations and routes of delivery are detailed herein.

Thus, one of skill in the art would readily appreciate that any one or more of the compositions described above, including the many specific embodiments, can be used by applying standard pharmaceutical manufacturing procedures to prepare medicaments to treat the many disorders described herein. Such medicaments can be delivered to the subject by using delivery methods known in the pharmaceutical arts.

### Administration of Additional Therapeutic Agents

The methods disclosed herein can further comprise, or alternatively consist essentially of, or yet further consist of administration of an effective amount of additional therapeutic agents to augment or enhance the therapeutic efficacy of the disclosed methods.

### EXAMPLES

### Example 1: Inhibition of NR3B to Block Development of Fatty Liver Disease

### Materials and Methods

Animals

Pten^{loxP/loxP}; Alb-Cre⁺ (Pten-null, Pm) and Pten^{loxP/loxP}; Alb-Cre-(Control, Con) mice were reported previously.¹⁷ C57/B16 mice were used for the high carbohydrate (HCD) diet studies. All animals were housed in a temperature-, humidity-, light-controlled room (12-h light/dark cycle), allowing free access to food and water. All experimental procedures were conducted according to the Institutional Animal Care and Use Committee (IACUC) guidelines at the University of Southern California.
Cell Culture

Control and Pten null hepatocytes were isolated from livers of wild type and Pten-null. Hepatocytes were cultured in Dulbecco's modified Eagle's medium (DMEM, Mediatech) supplemented with 10% FBS (Atlas Biologicals), 5 μg/ml insulin (Sigma), and 10 ng/ml epidermal growth factor (Invitrogen). Huh 7 liver cancer cell line was cultured in DMEM supplemented with 10% FBS. Pten null hepatocytes were transfected with pGL4 luciferase construct (Promega) expressing cytochrome c (cyt c) promoter followed firefly luciferase and puromycin-resistant selection marker. Pten null hepatocytes stably expressing luciferase construct were subsequently selected by puromycin.
Reagents, Plasmids and siRNAs

Oligomycin, FCCP, and Rotenone were purchased from VWR. Wild type PTEN-expressing plasmids pSG5-wtPTEN and pSG5 empty vector were previously described (13). Two siNR3B targeting sequences are 5′-ATCGAGAGATAGTGGTCACCATCAG-3′ and 5′-ATCGAGAGATAGTGGTCACCATCAG-3′. Cytochrome c luciferase reporter construct was generated by cloning the cytochrome c promoter sequence (−686-+55) into the luciferase construct pGL4 (Promega).
Transfection

Plasmids and siRNAs were transfected using lipofectamine 2000 (Invitrogen) according to the manufacturer's instructions. 4 μg of DNA and 10 μl of lipofectamine were delivered into cells growing at 50% confluence in 60 mm dishes.
Luciferase Reporter Assay

Pm hepatocytes (1×10⁵ cells) stably expressing pCytc/−686Luc luciferase construct were plated in each well of 6-well plates. The cell lysate preparation and luciferase activity measurement were conducted according to the manufacturer's instructions (Promega, E1910).
H&E, Oil Red O staining and Triglyceride Content Determination

The methodologies were previously described.⁸ Briefly, harvest tissues were fixed in 10% Zn-formalin overnight before tissue processing. Tissue sections were stained for hematoxylin and eosin (H&E), and used for staining with Ki67 and TUNEL. For Oil Red O staining, harvest tissues were snap froze in liquid nitrogen and preserved in OTC medium. For triglyceride (TG), protein and RNA analysis, harvest tissues were snap froze in liquid nitrogen until processed for biochemical assays. Analysis of TG were performed following the modified manufacture instructions (Wako Biochemical).⁸
Western Blot

Cell lysate preparation and immunoblotting analysis were performed as described previously.¹³ Antibodies against PTEN and p-AKT were purchased from Cell Signaling Technology. Antibody against NR3B was purchased from Abcam. Anti-actin was obtained from Sigma.
Seahorse XF24 Metabolic Flux Analysis

Cells were cultured on XF24 plates during experiment. The mitochondrial inhibitors, oligomycin (1 μM), FCCP (0.1 μM) and Rotenone (1 μM) were added after four measurements of basal OCR as described previously.¹⁴
RNA Isolation, Reverse Transcription and Quantitative PCR

Total RNA was isolated using Trizol reagent from Invitrogen. Reverse transcription was conducted using the reverse transcription system from Promega. Quantitative PCR was performed using SYBR green qPCR mix (Thermo) and 7900 HT fast real-time PCR system (Applied Biosystems). Gene-specific primers are as follows, cytochrome c forward 5′-CCAGTGCCACACCGTTGAA-3′ and reverse 5′-TCCCCAGATGATGCCTTTGTT-3′; Fas forward 5′-AGCGGCCATTTCCATTGCCC-3′ and reverse 5′-CCATGCCCAGAGGGTGGTTG-3′; ACC forward 5′-ACAGTGGAGCTAGAATTGGAC-3′ and reverse 5′-ACTTCCCGACCAAGGACTTTG-3′; MCAD forward 5′-TGTGGAGGTCTTGGACTTGGA-3′ and reverse 5′-TCCTCAGTCATTCTCCCCAAA-3′; GAPDH forward 5′-GCACAGTCAAGGCCGAGAAT-3′ and reverse 5′-GCCTTCTCCATGGTGGTGAA-3′.
Polyamide Treatment

25 nmole polyamide dissolved in 100 pi PBS supplemented with 5% DMSO was given per mouse every three days. Control group received 5% DMSO in 100 μl PBS per mouse.
Treatments Sustained for One Month
Statistical Analysis

Data in this example were presented as mean±SEM. Differences between individual groups were analyzed by the 2-tailed Student's t-test, and p≤0.05 was considered statistically significant.

### Inhibiting NR3B Binding to ERRE Blocks Fatty Liver Development Induced by Pten Ioss

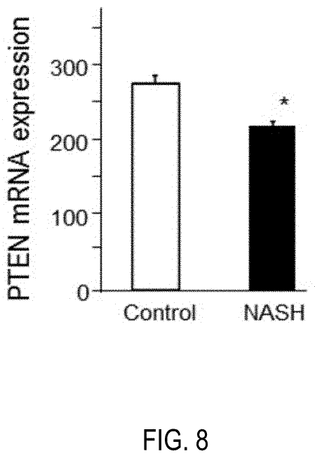

Mitochondrial abnormalities have been frequently reported to associate with development and progression of liver diseases. In the Pten null liver where steatosis develops, enhanced ROS and scavenger enzyme production associated with enhanced mitochondrial function was observed. This enhanced mitochondria function appears to result from increased mitochondrial biogenesis due to increased activity of NR3B5. In addition, PTEN expression is negatively correlated with NASH (FIG. 8). To test whether the NR3B inhibition could affect fatty liver development upon Pten deletion, a sequence specific small molecule polyamide to block binding of NR3B to the promoters of its target genes was developed. Pyrrole-imidazole polyamides are programmable DNA minor groove-targeting oligomers that bind with high affinity and specificity to the predetermined DNA sequences. Sequence specificity is achieved by programing the side-by-side pairings of pyrrole (Py) and imidazole (Im) to recognize the different hydrogen bonding patterns of nucleotides in the minor groove. The polyamide that was designed targets the consensus DNA sequence for NR3B 5′-TCAAGGTCA-3′, termed estrogen-related response element (ERRE)24. Gel mobility shift assay with biotin-labeled DNA probe containing ERRE showed that the polyamide that was designed (FIG. 1A) shares the same DNA binding sequence occupancy as NR3B (FIG. 1B).

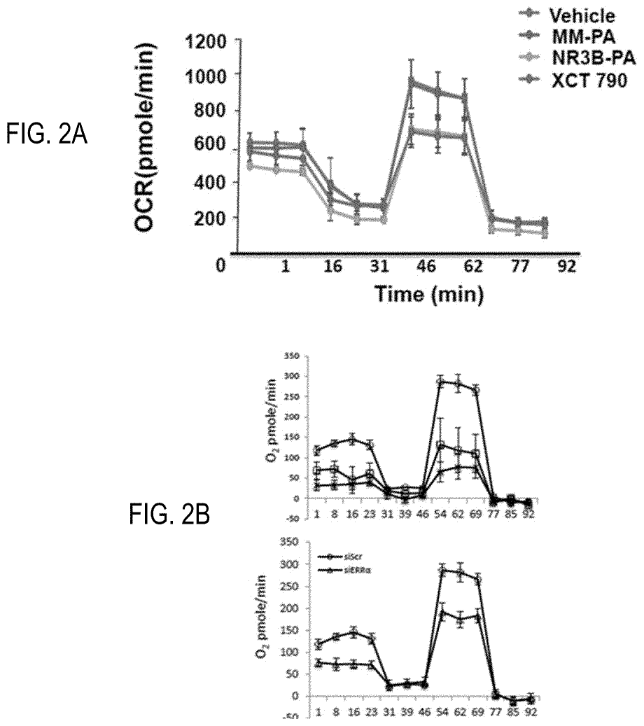

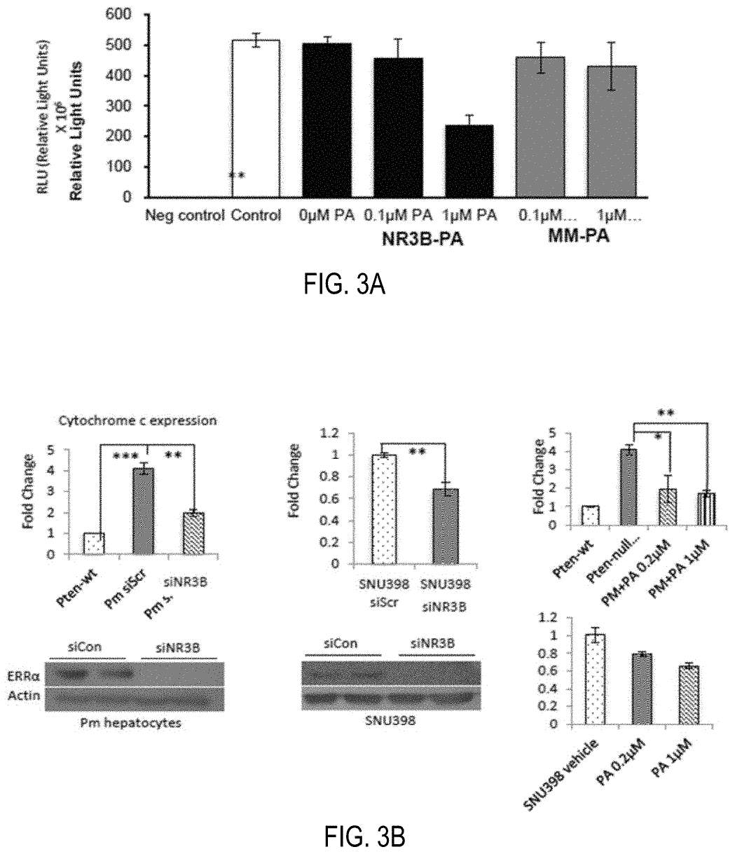

At the minimum effective dose for DNA Gel mobility shift assay (0.1 μM), NR3B-PA reduced both basal and maximal oxygen consumption rate (OCR) in a Seahorse assay whereas the control PA not matched to the sequence (MM-PA) did not (FIG. 2A). Increasing the dose of PA led to dose dependent reduction in OCR, similar to what is observed with knockdown of NR3B with siRNA (FIG. 2B). Using luciferase reporter containing the DNA binding element for NR3B, the NR3B-PA treatment inhibited the luciferase activity of the reporter whereas MM-PA did not at the same dose (FIG. 3A). In both mouse and human hepatocytes, NR3B-PA treatment downregulated the expression of cytochrome C, a target gene of NR3B (FIG. 3B). In addition, RNA seq analysis shows that more than 90% of the genes downregulated by NR3B-PA treatment are also regulated by siNR3B, confirming specificity of NR3B-PA for NR3B (FIG. 3C).

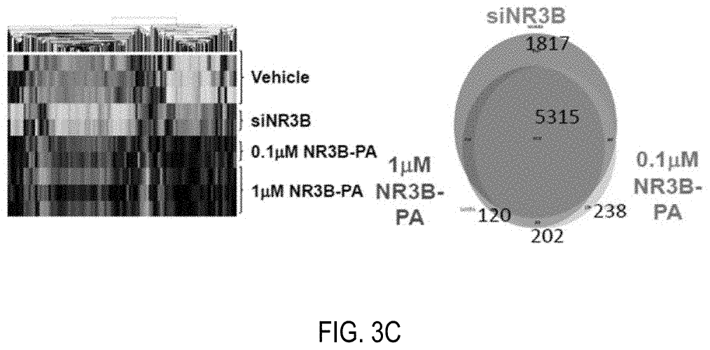

Using this NR3B-PA to block NR3B transcriptional activity, a 1.5-month old Pten-null mice was treated with 25 nmole polyamide every four days for 1 month and showed that inhibiting NR3B with polyamide blocked liver steatosis (FIG. 4A). The body weight of NR3B-PA-treated Pten null mice did not significantly diverge from the vehicle treated mice, indicating a limited toxicity of NR3B-PA to the Pten null mice (FIG. 4B). Morphology analysis of liver section showed massive lipid depositions in the Pten null livers whereas control livers maintained normal hepatic parenchymal without visible lipid droplets (FIG. 4A). Strikingly, NR3B-PA treatment in Pten null mice completely reversed the fatty liver phenotype, as evidenced by the minimal fat deposition observed in H&E sections (FIG. 4A). Oil Red O staining confirmed the increased lipid formation in Pten null livers and the dramatically reduced Oil Red O staining signals in NR3B-PA treated Pten null livers (FIG. 4A The hepatic lipid content was also quantified. Consistent with the morphological analysis, hepatic triglyceride (TG) quantification showed that NR3B-PA treatment remarkably rescued the elevated TG content observed in Pten null livers, to a level that is even lower than that of control mice (FIG. 4B).

Immunoblotting analysis using liver extracts from control, vehicle treated and NR3B-PA treated Pten null mice revealed that the level of NR3B protein is decreased in the liver upon NR3B-PA treatment (FIG. 4C). NR3B has been shown to act as a transcriptional activator of its own gene.¹² Thus, a decrease in NR3B protein levels is indicative that NR3B transcriptional activity is attenuated. This was confirmed using expression of cyt C and MCAD, two well characterized targets of NR3B transcriptional activity. Expressions of cyt C and MCAD are both higher in the Pten null liver tissues and reduced in livers extracted from err-PA treated mice (FIG. 4D). In the NR3B-PA treated groups, the expression levels of both genes are similar to that of controls where PTEN is intact. The qPCR analyses also indicated a downregulation of the mRNA expression for two of the key lipogenic enzymes, FAS and ACC are both reduced with NR3B-PA treatment when compared with vehicle treated Pten null mice. Expression of ACC is reduced by more than 30% while FAS expression is reduced by approximately 15%. Thus, NR3B inhibition by NR3B-PA blocked the fatty liver phenotype associated with PTEN loss.
NR3B Inhibition Blocks Biosynthesis of Triglycerides (Through RXR)

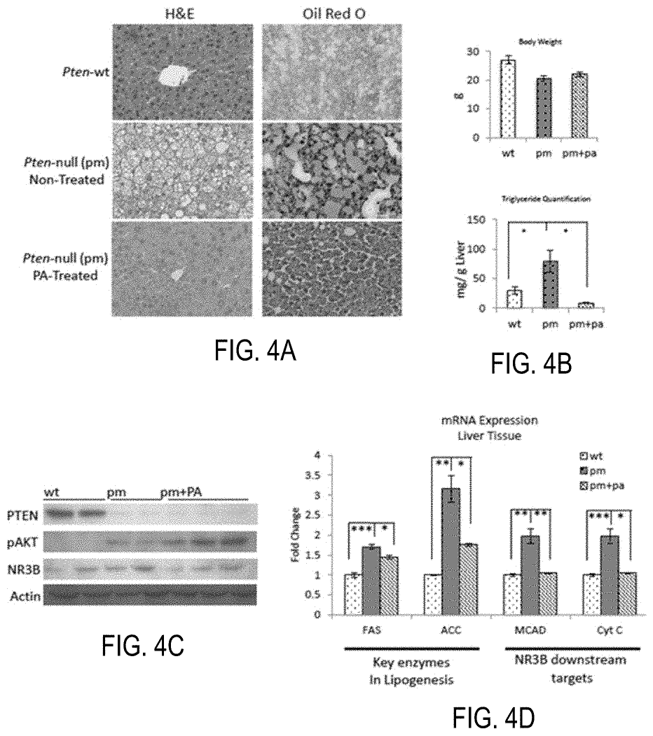

To explore the signals that may be responsible for the phenotype associated with NR3B inhibition, a RNA-seq analysis was performed and found that blocking NR3B activity using siNR3B led to downregulation of genes involved in triglyceride biogenesis. Biosynthesis of triglyceride starts with the acylation of glycerol-3-phosphate by two fatty acyl-CoA molecules. The product, phosphatidic acid is then converted to diacylglycerol through hydrolysis of the phosphate group followed by acylation with the third fatty acyl-CoA to form triglyceride. The RNA-seq data shows that all enzymes involved in the formation of triglyceride are downregulation as a result of siNR3B introduction into hepatocytes (FIG. 5). The results with qPCR analysis showing downregulation of DGAT2 and AGAPT3 with siNR3B treatment was confirmed (FIG. 5B). NR3B-PA treatment also induced similar downregulation of these genes in hepatocytes (FIG. 5C). In addition, the observation in vivo was also confirmed (FIG. 4D) that ACC and Fasn, two rate-limiting enzymes involved in lipogenesis are also downregulated with NR3B siRNA. These data suggest that NR3B inhibition may lead to reduced lipid accumulation in the liver through inhibition of de novo lipogenesis and triglyceride synthesis.

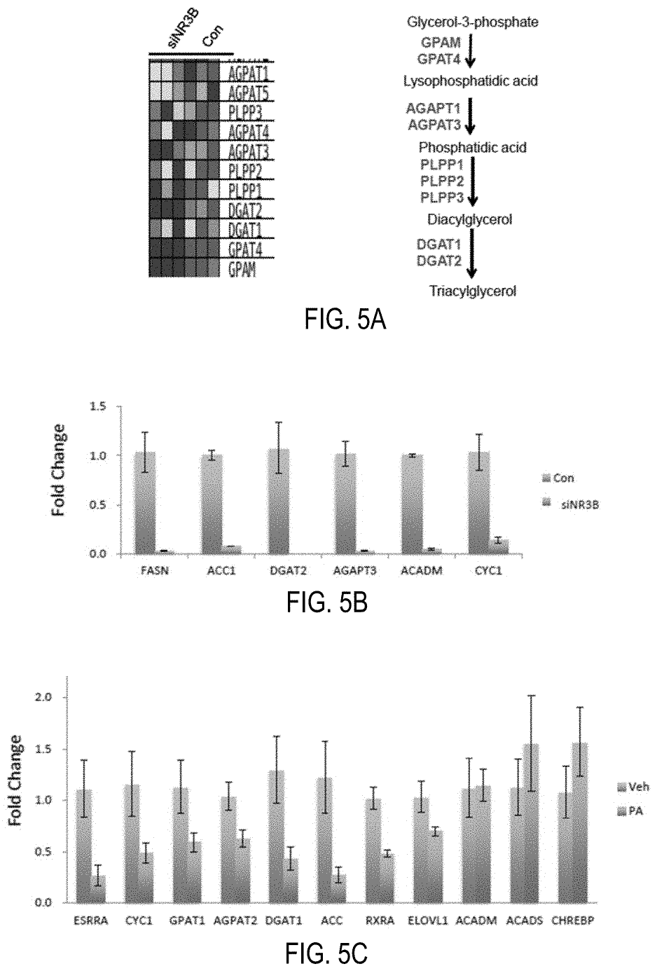

To address how NR3B may inhibit the lipogenic and triglyceride synthesis process, the RNA-seq data for SREBP and Chrebp, two transcriptional factors directly involved in the biosynthesis of lipid was explored. NR3B inhibition did not appear to affect srebp expression and Chrebp is upregulated as a result of siNR3B introduction and NR3B-PA treatment. An interesting observation is the downregulation of RXR with siNR3B and NR3B-PA (FIG. 6A). RXR binds to both PPARg and LXR and has the potential to regulate lipid metabolism through its interaction with these two nuclear receptors. It was explored whether RXR activation alters the expression of the lipogenic and triglyceride biosynthetic genes. This data indicates that treatment with Bexrotene, an agonist for RXR leads to induction of Fasn, DGAT2 and AGPAT3. This effect is unlikely to be a result of RXR' s role in mitochondrial function as Bexarotene treatment did not block the effects of NR3B-PA treatment on OCR production (FIG. 6B).
NR3B Inhibition Rescues NAFLD/NASH Development

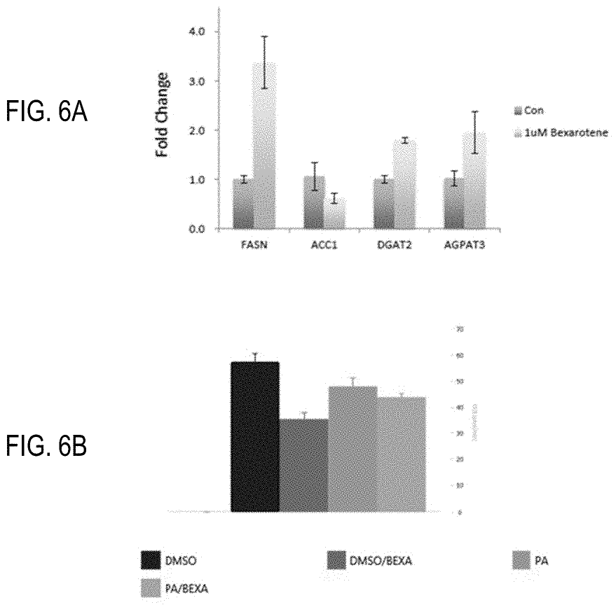

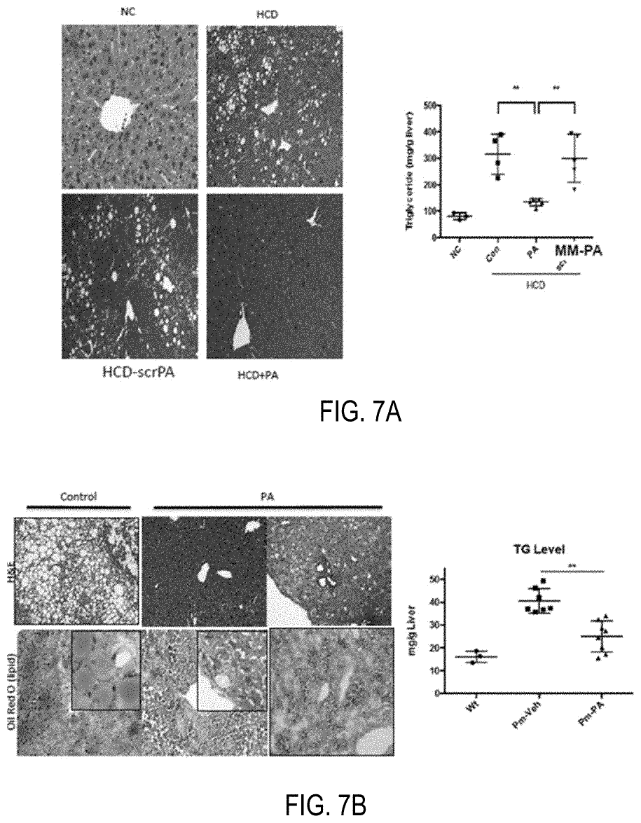

As NR3B inhibition appears to block the de novo synthesis of fatty acid and triglyceride, the effects of inhibiting NR3B on blocking NAFLD development in a model driven by high carbohydrate (HCD) feeding were explored. In this model, lipid accumulation in the liver is induced by feeding of HCD for 4 months. HCD led to 3 fold increase of triglyceride in the liver was shown. Treatment with NR3B-PA led to significant inhibition of lipid accumulation. Liver triglyceride levels in the NR3B-PA group is comparable to that of the normal chow fed mice (FIG. 7A). Mismatched PA (MM-PA) is not able to reduce lipid accumulation in the liver of the HCD fed mice, confirming that the effects of NR3B-PA is specific to NR3B inhibition.

To further explore the effects of NR3B inhibition on reversing the development of fatty liver, particularly at the NASH stage, The 7.5 months old Pten null mice was treated with NR3B-PA using the same treatment protocol. These mice developed not only NAFLD but also inflammatory cell infiltration, resembles NASH conditions. Similar to the 1.5-months old mice, NR3B-PA treatment reversed the severe NASH phenotype that is observed with PTEN loss (FIG. 5A).

### Equivalents

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

Unless otherwise defined, all technical and scientific terms used herein have the same meaning as commonly understood by one of ordinary skill in the art to which this disclosure belongs. All nucleotide sequences provided herein are presented in the 5′ to 3′ direction.

The embodiments illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure.

Thus, it should be understood that although the present disclosure has been specifically disclosed by specific embodiments and optional features, modification, improvement and variation of the embodiments therein herein disclosed may be resorted to by those skilled in the art, and that such modifications, improvements and variations are considered to be within the scope of this disclosure. The materials, methods, and examples provided here are representative of particular embodiments, are exemplary, and are not intended as limitations on the scope of the disclosure.

The scope of the disclosure has been described broadly and generically herein. Each of the narrower species and subgeneric groupings falling within the generic disclosure also form part of the disclosure. This includes the generic description with a proviso or negative limitation removing any subject matter from the genus, regardless of whether or not the excised material is specifically recited herein.

In addition, where features or aspects of the disclosure are described in terms of Markush groups, those skilled in the art will recognize that embodiments of the disclosure may also thereby be described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control. Throughout this disclosure, various publication are referenced by a citation, the full bibliographic citation for each are provided immediately preceding the claims.

### REFERENCES

1 Wong R J, Aguilar M, Cheung R, et al. Nonalcoholic steatohepatitis is the second leading etiology of liver disease among adults awaiting liver transplantation in the United States. Gastroenterology. 2015; 148: 547-555.

2 Smits M M, Ioannou G N, Boyko E J, Utzschneider K M. Non-alcoholic fatty liver disease as an independent manifestation of the metabolic syndrome: results of a US national survey in three ethnic groups. J Gastroenterol Hepatol. 2013; 28: 664-670.

3 Younossi Z M, Koenig A B, Abdelatif D, Fazel Y, Henry L, Wymer M. Global epidemiology of nonalcoholic fatty liver disease-Meta-analytic assessment of prevalence, incidence, and outcomes. Hepatology. 2016; 64: 73-84.

4 Neuschwander-Tetri B A, Loomba R, Sanyal A J, et al. Farnesoid X nuclear receptor ligand obeticholic acid for non-cirrhotic, non-alcoholic steatohepatitis (FLINT): a multicentre, randomised, placebo-controlled trial. Lancet. 2015; 385: 956-965.

5 Koo S H. Nonalcoholic fatty liver disease: molecular mechanisms for the hepatic steatosis. Clin Mol Hepatol. 2013; 19: 210-215.

6 Samuel V T Shulman G I. Nonalcoholic Fatty Liver Disease as a Nexus of Metabolic and Hepatic Diseases. Cell Metab. 2018; 27: 22-41.

7 Garcia-Ruiz C, Baulies A, Mari M, Garcia-Roves P M, Fernandez-Checa J C. Mitochondrial dysfunction in non-alcoholic fatty liver disease and insulin resistance: cause or consequence? Free radical research. 2013; 47: 854-868.

8 Begriche K, Massart J, Robin M A, Bonnet F, Fromenty B. Mitochondrial adaptations and dysfunctions in nonalcoholic fatty liver disease. Hepatology. 2013; 58: 1497-1507.

9 Hirschey M D, Shimazu T, Goetzman E, et al. SIRT3 regulates mitochondrial fatty-acid oxidation by reversible enzyme deacetylation. Nature. 2010; 464: 121-125.

10 Ibdah J A, Paul H, Zhao Y, et al. Lack of mitochondrial trifunctional protein in mice causes neonatal hypoglycemia and sudden death. J Clin Invest. 2001; 107: 1403-1409.

11 Blanpain CFuchs E. Stem cell plasticity. Plasticity of epithelial stem cells in tissue regeneration. Science. 2014; 344: 1242281.

12 Knutti DKralli A. PGC-1, a versatile coactivator. Trends in endocrinology and metabolism: TEM. 2001; 12: 360-365.

13 Luo J, Sladek R, Carrier J, Bader J A, Richard D, Giguere V. Reduced fat mass in mice lacking orphan nuclear receptor estrogen-related receptor alpha. Molecular and cellular biology. 2003; 23: 7947-7956.

14 Debebe A, Medina V, Chen C Y, et al. Wnt/beta-catenin activation and macrophage induction during liver cancer development following steatosis. Oncogene. 2017; 36: 6020-6029.

15 Galicia V A, He L, Dang H, et al. Expansion of hepatic tumor progenitor cells in Pten-null mice requires liver injury and is reversed by loss of AKT2. Gastroenterology. 2010; 139: 2170-2182.

16 He L, Hou X, Kanel G, et al. The critical role of AKT2 in hepatic steatosis induced by PTEN loss. The American journal of pathology. 2010; 176: 2302-2308.

17 Stiles B, Wang Y, Stahl A, et al. Liver-specific deletion of negative regulator Pten results in fatty liver and insulin hypersensitivity [corrected]. Proceedings of the National Academy of Sciences of the United States of America. 2004; 101: 2082-2087.

18 Huch M, Dorrell C, Boj S F, et al. In vitro expansion of single Lgr5++liver stem cells induced by Wnt-driven regeneration. Nature. 2013; 494: 247-250.

What is claimed is:

1. A method for one or more of: inhibiting the development of non-alcoholic steatohepatitis (NASH), non-alcoholic liver steatosis (NAFLD), fatty liver disease, liver fibrosis, hepatocellular carcinoma; blocking the biosynthesis of fatty acids and triglycerides; inhibiting de novo lipogeneis; the method comprising administering an effective amount of an agent that interferes with the binding of the estrogen receptor related receptor (NR3B) to a NR3B target to a subject in need thereof.

2. The method of claim 1, wherein the NR3B target are the promoters of a NR3B target gene.

3. The method of claim 2, wherein the NR3B target gene is cytochrome C or MCAD.

4. The method of claim 1, wherein the agent is selected from the group of: a small molecule, an anti-NR3B antibody, an NR3B-polyamide (NR3B-PA), an anti-NR3B inhibitory RNA, or siNR3B.

5. The method of claim 1, wherein the subject is a mammal.

6. The method of claim 5, wherein the mammal is a human.

7. The method of claim 1, wherein the agent is NR3B-PA or an equivalent thereof or siNR3B or an equivalent thereof.

8. The method of claim 1, wherein the administration is in one or more doses.

9. The method of claim 1, wherein the administration is local to the liver or systemic.

10. The method of claim 1, further comprising administering an agent that treats the relative one or more diseases or conditions.
